(19) United States
(12) Patent Application Publication
(10) Pub. No.: US 2022/0347208 A1
(43) Pub. Date: Nov. 3, 2022

(54) FORMULATIONS USING LITHIUM TO TREAT GOUT ARTHROPATHY

(71) Applicant: Carl Ganio, Wilmington, NC (US)

(72) Inventor: Carl Ganio, Wilmington, NC (US)

(21) Appl. No.: 17/621,236

(22) PCT Filed: Jun. 19, 2020
(86) PCT No.: PCT/US2020/038736

§ 371 Date: Dec. 20, 2021

Related U.S. Application Data
(60) Provisional application No. 62/863,709, filed on Jun. 19, 2019.
(60) Provisional application No. 62/991,200, filed on Mar. 18, 2020.
(60) Provisional application No. 63/026,302, filed on May 18, 2020.

(51) Int. Cl. A61K 33/14; A61K 31/609; A61P 19/02; A61K 47/20; A61K 47/28; A61K 47/68; A61K 38/00

(52) U.S. Cl. CPC A61K 33/14; A61K 31/609; A61P 19/02; A61K 47/20; A61K 47/28; A61K 47/6805; A61K 38/005

(57) ABSTRACT

Disclosed are lithium-based formulations and methods of using the same for the prevention and treatment of inflammatory conditions, gout, joint disease, pain and symptoms thereof.

### CROSS-REFERENCE TO RELATED APPLICATIONS

This application claims priority to and the benefit of U.S. Patent Application Ser. No. 62/863,709, filed Jun. 19, 2019; U.S. Patent Application Ser. No. 62/991,200, filed Mar. 18, 2020 and U.S. Patent Application Ser. No. 63/026,302, filed May 18, 2020. The disclosures of each are hereby incorporated herein by reference in their entireties.

### FIELD

The present inventive concept relates generally to a formulation for the prevention and treatment of inflammatory conditions, gout, joint disease and pain, and symptoms thereof. More specifically, the present inventive concept provides formulations and methods using topical or injectable lithium to prevent or treat inflammatory conditions, gout, joint disease and pain, and symptoms thereof.

### BACKGROUND

Gout and pseudo gout are crystalline arthropathies. The formation and deposition of insoluble crystals (monosodium urate and calcium pyrophosphate) cause inflammatory reactions within the tissues, tendons and joint capsule of mammals. The incidence of gout has risen significantly and is projected to continue.

Current medications to treat acute gout attacks include nonsteroidal anti-inflammatory drugs (NSAIDs), colchicine and corticosteroids. Medications frequently used to prevent gout attacks and gout-related complications include xanthine oxidase inhibitors and uricosurics. However, these medications may affect hepatic and renal function, causing side effects. The use of these oral medications is preventative; and not intended to treat the painful or inflammatory symptoms that exist in situ. Xanthine oxidase inhibitors and uricosurics may not be useful for prodromal or vague inter-attack symptoms of quiescent gout.

Lithium is believed to demonstrate neuroprotective and anti-inflammatory effects. The basic chemistry of the lithium ion stems from its small anatomic size and its promiscuity as one of the most reactive of the alkali metals. It has the ability to block some ion channels and alter the flux in others by reacting in place of magnesium, calcium, potassium or sodium. Currently, lithium carbonate is approved by the United States Food and Drug Administration for the treatment of bipolar disorder and lithium remains the treatment of choice for Bipolar Disorder. However, lithium has a narrow safety range in the serum and is tightly regulated between 0.6-1.2 mEq/L. The risk of side effects and potentially fatal complications can occur at serum concentrations of ˜1.5-2.0 mEq/L and above. Understandably, lithium is not currently accepted as a standard treatment for other diagnoses given its propensity to illicit patient sensitivity, and more detrimentally, toxicity.

Although various methods and substances for treating pain exist, such methods are often an inconvenience in terms of pain management interventions, devices and surgical intervention, or the substances impair the patient's motor functions and/or can lead to tolerance, hypersensitivity, peripheral sensitization, central sensitization and/or addiction. Moreover, many of the treatments for acute pain may be addictive, and therefore, are not appropriate for long-term use as pain management therapy.

Thus, new gout treatments and adjunctive pain treatments that are less toxic, more affordable, readily available and non-addictive are needed.

### SUMMARY

The present inventive concept provides a method of preventing or treating an inflammatory condition, joint disease or symptoms thereof in a subject. The method includes administering to the subject an effective amount of a composition including lithium in a pharmaceutically acceptable carrier. In some embodiments, the inflammatory condition is joint disease, arthritis, tendonitis, bursitis, inflammation of the ligament, synovitis, gout, or systemic lupus erythematosus.

In further aspects, the number, duration, frequency, or intensity of a gout attack experienced by a subject is decreased during initiation or maintenance administration of the composition including lithium in a pharmaceutically acceptable carrier.

The present inventive concept also provides a method of preventing or treating acute or chronic pain. The method includes topical or parenteral administration of a composition including lithium in a pharmaceutically acceptable carrier to a subject in need thereof.

Additional aspects of the present inventive concept further provide methods of preventing or treating inflammatory conditions, inflammatory joint diseases as well as preventing or treating acute and chronic pain including administering to a subject a composition including lithium in a pharmaceutically acceptable carrier in combination with an anti-gout agent and/or a pain management agent.

The present inventive concept also provides lithium formulated with a pharmaceutically acceptable carrier, with or without additional active ingredients, pharmaceutical agents and/or transport enhancers. In some embodiments, the formulation is a topical or parenteral formulation including lithium and a pharmaceutically acceptable carrier.

According to another aspect of the present inventive concept, provided are kits for preventing or treating inflammatory conditions, inflammatory joint diseases as well as preventing or treating acute and chronic pain. The kits include lithium and components described herein packaged together with optional instructions (written, audio or visual) regarding how to use the components of the kit.

### BRIEF DESCRIPTION OF THE DRAWINGS

FIG. 1 depicts group summary data for ankle swelling in the efficacy evaluation of lithium carbonate on gouty inflammation in male Sprague Dawley rats.

FIG. 2 depicts group summary data for pain index in the efficacy evaluation of lithium carbonate on gouty inflammation in male Sprague Dawley rats.

FIG. 3 depicts IL-1β levels in synovial fluid from ankle joints of male Sprague Dawley rats in the efficacy evaluation of lithium carbonate on gouty inflammation.

FIG. 4 depicts IL-6 levels in synovial fluid from ankle joints of male Sprague Dawley rats in the efficacy evaluation of lithium carbonate on gouty inflammation.

FIG. 5 depicts KC-GRO levels in synovial fluid from ankle joints of male Sprague Dawley rats in the efficacy evaluation of lithium carbonate on gouty inflammation.

FIG. 6 depicts TNF-α levels in synovial fluid from ankle joints of male Sprague Dawley rats in the efficacy evaluation of lithium carbonate on gouty inflammation.

### DETAILED DESCRIPTION

The present inventive concept will now be described more fully hereinafter. The inventive concept, however, may be embodied in many different forms and should not be construed as limited to the exemplary embodiments as set forth herein. Rather, these embodiments are provided so that the disclosures will be thorough and complete, and will fully convey the scope of the inventive concept to one of ordinary skill in the art.

As used herein, “a,” “an,” or “the” can mean one or more than one. Also as used herein, “and/or” refers to and encompasses any and all possible combinations of one or more of the associated listed items, as well as the lack of combinations when interpreted in the alternative (“or”).

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items. Moreover, it should be noted that various embodiments of the presently disclosed subject matter may “comprise,” “consist” or “consist essentially of” the components or ingredients disclosed herein.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of any numerical ranges by endpoints or in succession includes all numbers, e.g., whole integers, including fractions thereof and decimals, subsumed within that range or sequence (for example, the recitation of 1 to 5 includes 1, 2, 3, 4, and 5, as well as fractions thereof, e.g., 1.5, 2.25, 3.75, 4.1, and the like and any range within that range and at least 95%, 96%, 97%, 98% or 99% includes all decimals in between and up to the next higher percentage up to 100).

The term “prevent,” “preventing” or “prevention of” (and grammatical variations thereof) refers to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the inventive concept. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression are less than what would occur in the absence of carrying out the steps of the methods of the present invention.

As used herein, the expression “treat,” “treating” “treatment of” (and grammatical variations thereof) means improving, reducing, or alleviating at least one symptom or biological consequence of the inflammatory condition, inflammatory joint disease or pain. The subject may exhibit or be diagnosed with one or more symptoms or biological consequences of an inflammatory condition, inflammatory joint disease or pain.

A “therapeutically effective amount,” “treatment effective amount” and “effective amount” as used herein are synonymous unless otherwise indicated, and mean an amount of a composition or formulation of the present inventive concept that is sufficient to improve the condition, disease, or disorder being treated and/or achieved the desired benefit or goals as described herein. Those skilled in the art will appreciate that the therapeutic effects need not be complete or curative, as long as some benefit is provided to the subject. Similarly, a “prevention effective” amount is an amount that is sufficient to prevent (as defined herein) the disease, disorder and/or clinical symptom in the subject. Those skilled in the art will appreciate that the level of prevention need not be complete, as long as some benefit is provided to the subject.

The presently disclosed subject matter provides methods for preventing or treating an inflammatory condition in a subject. The method includes administering to the subject an effective amount of a composition including lithium in a pharmaceutically acceptable carrier.

In some embodiments, the inflammatory condition is gout, pseudo gout, osteoarthritis, rheumatoid arthritis, psoriatic arthritis or systemic lupus erythematosus. In still other embodiments, the inflammatory condition is an inflammatory joint disease. In some embodiments, the inflammatory joint disease is arthritis, tendonitis, bursitis, inflammation of the ligament, synovitis or gout. In some embodiments, the inflammatory joint disease is gout.

Gout refers to a group of disorders or symptoms most often associated with the accumulation of uric acid due to an overproduction of uric acid or a reduced ability of the kidney to excrete uric acid. Gout is often characterized by the deposition of urate crystals (uric acid or salts thereof, e.g. monosodium urate) in the joints (gouty arthropathy) or soft tissue (tophi). Gout usually affects the large joint of the hallux (“great toe” or “big toe”), but it can occur in any joint. Other commonly affected joints include the knee, elbow, wrist, finger, foot, ankle, hip, shoulder and spine. If gout is not actively prevented or treated properly, gout may worsen with more joints attacked and/or gout attacks occurring more frequently. As used herein, “gout” may refer to specific classifications and causations of gout as well as gout-related disorders such as acute gout, chronic gout, moderate gout, severe gout, recurrent gout, refractory gout, gouty arthritis, hyperuricaemia, gout-related cardiovascular disorders, Lesch-Nyhan syndrome, Kelley-Seegmiller syndrome, kidney disease, kidney stones, kidney failure, joint inflammation, inflammatory joint disease, arthritis, osteoarthritis, rheumatoid arthritis and psoriatic arthritis, crystalline arthropathy, bursitis, tendinopathy urolithiasis, plumbism, hyperparathyroidism, psoriasis or sarcoidosis. “Acute gout” refers to gout present in a subject with at least one gouty symptom (e.g., podagra or other gouty arthritis, gout flare, gouty attack). “Chronic gout” refers to gout present in a subject having recurrent or prolonged gout flares, tophus formation, chronic inflammatory arthritis, or joint deterioration associated with gout, and includes the periods following recovery from acute gout and between acute gout attacks (i.e., intercritical gout). “Moderate gout” refers to gout present in a subject having at least two gout flares in the past 12 months. “Severe gout” refers to gout present in a subject having tophaceous deposits in the joints, skin, or kidneys resulting in chronic arthritis, joint destruction, subcutaneous tophi, or kidney dysfunction, and, in some cases, with subsequent deformity and/or disability. “Recurrent gout” refers to subjects experiencing gout attacks or flare-ups several times a year. “Refractory gout” refers to gout in patients who are unresponsive or poorly responsive to one or more second urate-lowering agents, or have experienced or are at an increased risk of experiencing an adverse event therefrom. The terms “unresponsive” and “poorly responsive” in this context include (1) no or insignificant lowering of serum uric acid, (2) failure to reach a target serum uric acid level (e.g., as determined by a physician or other medical practitioner), and (3) the persistence of one or more gouty conditions or symptoms such as gout flares, gouty tophus, gouty arthritis, or other associated conditions regardless of any lowering of serum uric acid levels.

In particular embodiments, lithium, an active ingredient in the formulations of the present inventive concept, includes, but is not limited to, elemental lithium, lithium ion, a lithium salt, a lithium derivative, etc. In some embodiments, lithium is elemental lithium, a lithium mimetic, lithium acetate, lithium aluminate, lithium aluminum hydride, lithium amide, lithium aspartate, lithium azide, lithium beryllide, lithium bis(trifluoromethanesulfonyl)imide, lithium bis(trimethylsilyl)amide, lithium borate, lithium borohydride, lithium bromide, lithium carbide, lithium carbonate, lithium chlorate, lithium chloride, lithium citrate, lithium cobalt oxide, lithium cyanide, lithium diisopropylamide, lithium disilicate, lithium fluoride, lithium hexafluorogermanate, lithium hexafluorophosphate, lithium hydride, lithium hydroxide, lithium hypochlorite, lithium imide, lithium iodate, lithium iodide, lithium iridate, lithium iron phosphate, lithium lactate, lithium metaborate, lithium metasilicate, lithium methoxide, lithium molybdate, lithium molybdenum purple bronze, lithium monoxide anion, lithium nickel cobalt aluminum oxides, lithium nickel manganese cobalt oxides, lithium niobate, lithium nitrate, lithium nitride, lithium nitrite, lithium orotate, lithium orthosilicate, lithium oxide, lithium perchlorate, lithium peroxide, lithium platinate, lithium polonide, lithium ruthenate, lithium salicylate, lithium selenide, lithium stearate, lithium succinate, lithium sulfate, lithium sulfide, lithium sulfite, lithium superoxide, lithium tantalate, lithium tetrachloroaluminate, lithium tetrafluoroborate, lithium tetrahydridogallate, lithium tetrakis(pentafluorophenyl)borate, lithium tetramethylpiperidide, lithium titanate, lithium triborate, lithium triethylborohydride, lithium triflate or lithium tungstate. In some embodiments, lithium is lithium carbonate, lithium citrate, lithium salicylate or lithium lactate. In particular embodiments, lithium is lithium carbonate. In some embodiments, the lithium in any combination or formulation, may penetrate to the capsular, tendinous, and periarticular structures in sufficient concentration to produce the desired effect without systemic toxicity. Moreover, the number, duration, frequency, and/or intensity of a gout flare experienced by a subject is decreased during initiation or maintenance administration of the composition including lithium in a pharmaceutically acceptable carrier to a subject. Additionally, according to embodiments of the present inventive concept, lithium may be used for at least prevention of gouty attacks in susceptible individuals, treatment of acute gouty inflammatory attack, treatment of quiescent or indolent gout, resolution of gouty tophus and treatment of calcium pyrophosphate crystal deposition disease.

In further embodiments, the composition including lithium is administered topically or parenterally to the subject. “Topical administration” as used herein refers to application onto the surface of keratinous tissue such as skin, nail, mucosa, etc. and encompasses transdermal administration. Such compositions are typically dermatologically-acceptable in that they do not have undue toxicity, incompatibility, instability, allergic response, and the like, when applied to skin. In particular embodiments, the composition including lithium is applied to any anatomically penetrable superficial area of the subject's body. In some embodiments, the composition including lithium is applied to a knee, calf, elbow, wrist, finger, toe, foot, ankle, hip, shoulder and/or spine of a subject.

The use of the compositions including lithium for topical administration includes use in solution, liquid, spray, foam, gel, paste, cream, ointment, lotion, serum, oil, liniment, aerosol, roll-on liquid, sheet, patches (including microneedle patches and gel-based patches), iontophoresis, etc. Additionally, the topical composition can be impregnated into or used as a coating on a bandage, patch, adhesive material, support wrap, glove, sock, footwear, fabric, or similar article for ease of application. In particular embodiments, the topical formulation is a cream. In accordance with the subject matter described herein, a topical dosage form may be packaged in, for example, a multi-use or single-use package, including for example, a tube, a bottle, a pump, a container or bottle, a vial, a jar, a packet, or a blister package, etc.

The topical formulations of the present inventive concept may remain stable in storage at standard room temperature for periods including up to about 5 years, between about 3 months and about 5 years, between about 3 months and about 4 years, between about 3 months and about 3 years, and alternately any time period between about 6 months and about 3 years.

For parenteral administration, the disclosed compositions including lithium may be formulated for injection or local infusion, for example, intravenous (short-term), intramuscular or subcutaneous injection or infusion, or for administration in an anatomically-localized bolus or push dose, delayed release or continuous infusion. In particular embodiments, the parenteral administration does not include intravenous administration. In some embodiments, where the parenteral administration is intravenous, the intravenous injection is a rapid injection, push or bolus. In some embodiments, where the parenteral administration is an infusion, the infusion is a local infusion. Suspensions, solutions, gels or emulsions in an oily or aqueous vehicle, optionally containing other formulatory agents such as suspending, stabilizing or dispersing agents may be used.

Injections may be made into the intra-articular space, peri-articular space, soft tissues, lesions, epidural space, perineural space, or the foraminal space at or near the site of a patient's discomfort, trigger point or painful pathology. In some embodiments, the injection is made into an inflamed joint. In some embodiments, the composition including lithium is not injected into the joint capsule. In some embodiments, the composition including lithium is injected around the outside of the joint and/or bursa in order to bathe extra-capsular structures. In particular embodiments, the compositions including lithium are delivered as a single injection or as sequential injections. In certain embodiments, the composition including lithium is injected into or near an anatomically penetrable superficial area of the subject's body. In some embodiments, the area is a knee, calf, elbow, wrist, finger, toe, foot, ankle, hip, shoulder and/or spine of a subject. In some embodiments, the injections are subcutaneous having a depth in the range of about 3-7 mm, about 3-5 mm or not exceeding about 5 mm.

In particular embodiments, the topical or parenteral formulations including lithium may be an immediate release form or may provide a controlled or sustained release of the drug at the site of administration. In some embodiments, the topical or parenteral formulations may be microdosed or microdispensed where the lithium dose is unlikely to produce whole-body effects, but high enough to allow the cellular response to positively affect inflammatory condition, inflammatory joint disease, pain or symptoms thereof. In particular embodiments, the compositions including lithium according to the present inventive concept show no detectable levels of serum lithium, particularly in standard laboratory sensitivity range. Accordingly, there is a decreased chance of lithium toxicity associated with topical or parenteral lithium administration compared to conventional lithium pharmaceutical compositions. Further, topical or parenteral lithium administration may allow serum uptake at sub-therapeutic levels.

In certain embodiments, in addition to the lithium, the topical formulations and/or transdermal therapeutic systems of the present inventive concept may include at least one adjuvant such as a penetration enhancer, anti-oxidant, stabilizer, carrier, or vehicle. Additionally or alternatively, the present inventive concept may include the application of electric current (iontophoresis) for enhancing permeation of the lithium.

Suitable penetration enhancers useful in the formulations of the present inventive concept include, but are not limited to, isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, diethylene glycol monoethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and terpenes.

In certain embodiments, the compositions including lithium and the pharmaceutically acceptable carrier will typically contain on the order of about 0.001 to about 99.9% by weight lithium, in some embodiments, 0.01 wt. % to 90 wt. % lithium, 1 wt. % to about 80.0 wt. %, 1 wt. % to about 50.0 wt. %, or about 1 wt. % to about 30 wt. % lithium, with the remainder of the composition including a pharmaceutically acceptable carrier and/or an additional therapeutic agent for gout and/or pain. As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the inventive concept, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as mineral oil, peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; sterile water; injection grade (WFI) (highly purified waters containing less than 10 CFU/100 ml of aerobic bacteria); deionized; distilled water; isotonic saline; Ringer's solution; dimethyl sulfoxide (DMSO); ethyl alcohol; phosphate buffer solutions; hydrogenated polyisobutene (and) ethylene/propylene/styrene copolymer (and) butylene/ethylene/styrene copolymer (Versagel®); acetic acid; and other non-toxic compatible substances employed in pharmaceutical formulations.

Embodiments of the present inventive concept further include concomitant administration to the subject of an effective amount of an anti-gout agent or a pain management agent along with administration of the composition including lithium in a pharmaceutically acceptable carrier. The terms “concomitant administration”, “concomitantly administering” and “combination therapy” refer to the administration of two or more agents in any manner in which the pharmacological effects of those agents are manifested in the subject at the same time. These terms encompass administering two or more agents to a subject substantially concurrently, for example in a single dosage form (e.g. a solution, spray, foam, gel, paste, cream, ointment, lotion, serum, oil, liniment, aerosol, liquid, sheet, patch, ampule, syringe, etc.), administering at least one agent in one dosage form and the other agent(s) in a separate dosage form, and administering each agent in its own separate dosage form. The administration may be performed sequentially or simultaneously. For example, for sequential administration, the first agent may be administered before or after the second agent. In some embodiments, concomitant administration includes concurrent administration. In other embodiments, concomitant administration includes sequential administration. In particular embodiments, the anti-gout agent or the pain management agent concomitantly administered with the composition including lithium in a pharmaceutically acceptable carrier is a nonsteroidal anti-inflammatory drug (NSAID), an opiate, a local anesthetic, a steroid, a xanthine oxidase inhibitor, a uricosuric drug, or an alkaloid drug. In further embodiments, the anti-gout agent or the pain management agent concomitantly administered with the composition including lithium in a pharmaceutically acceptable carrier is allopurinol, probenecid, benzbromarone, sulfinpyrazone, febuxostat, lesinurad, pegloticase, indomethecin or colchicine.

According to further embodiments of the present inventive concept, lithium treatment as described herein may be an option for the prevention or treatment of pain. Pain is often a sign of acute inflammation. Pain may be divided into two general categories—acute and chronic. Acute pain is typically characterized by rapid its onset, intensity, and short duration. Chronic pain, however, tends to be persistent, such as pain associated with inflammation, arthritis, etc. Chronic pain may also cause individuals to exhibit enhanced sensitivity to painful stimulus (hyperalgesia); painful sensation to normally non-painful stimulus (allodynia); burning sensation; and unusual nociceptive descriptors (stabbing, sharp, throbbing, etc.). In addition, chronic pain may also have additional physiological consequences such as trigger point producing pain (myofascial pain or radicular pain) or sympathetic dystrophy (warm/cold extremities, joint stiffness, or bone demineralization). Accordingly, compositions including lithium in a pharmaceutically acceptable carrier can be used to prevent or treat nociceptive; neuropathic; phantom; psychogenic; breakthrough pain; incident pain; pain caused by inflammation of joints, tendons, nerves, muscle, and other soft tissues; arthritic pain; back pain; headache pain; and pain caused by cancer. In particular embodiments, the pain is acute pain, chronic pain, nociceptive pain and neuropathic pain. In some embodiments, the pain is complex regional pain syndrome (CRPS), allodynia, fibromyalgia, diabetic neuropathy, chemotherapy pain, shingles, trigeminal neuralgia or thoracic post herpetic neuralgia.

In some embodiments, lithium treatment as described herein may be a replacement for, or minimize the use of, opioid or narcotic pain medications including, but not limited to fentanyl; hydrocodone; hydrocodone and acetaminophen; hydromorphone; meperidine; methadone; morphine; oxycodone; oxycodone and acetaminophen; and oxycodone and naloxone. According to embodiments of the present inventive concept, the lithium compositions described herein may be used for at least the following: lithium alone or in combinations for topical/transdermal treatment of hyperalgesia, allodynia, neuropathy, nerve pain, neuritis/neuralgia; lithium injection or transdermal use for dorsal root ganglion application; lithium injection for chronic pain or acute spinal injury (for neuroprotection); and lithium injection in combination with local anesthetic (plain or liposomal) to provide extended nerve block, and to reduce the effective dose.

In particular embodiments of the present inventive concept, topical lithium, and in particular, lithium carbonate, has potential for the management of peripheral nerve pain of many etiologies. Topical use may allow delivery to the skin, nociceptors, afferent nerves, dermis, bursae, joint and tendon. A topical lithium composition, such as lithium carbonate, may act on inflammation, acute pain, and chronic pain. An intradermal, subcutaneous or periarticular injection of lithium may arrest an acute gout attack.

The use of lithium compositions as described herein with a local anesthetic may provide longer duration nerve block for procedures. The use of lithium compositions as described herein in a liposomal local anesthetic vehicle may potentially extend postoperative relief of pain beyond the 48-72 hours provided by the liposomal anesthetic alone. Further, the use of injectable lithium to or around the dorsal root ganglion via intervertebral block may mitigate the effects of CRPS, allodynia, fibromyalgia, diabetic neuropathy, chemotherapy pain, shingles, etc. Local blocks may treat trigeminal neuralgia. Intercostal blocks may treat thoracic post herpetic neuralgia.

Notably, topical use of the compositions including lithium in a pharmaceutically acceptable carrier may allow delivery to the skin, afferent nerves, dermis, bursae, joint and tendon. In some instances, topical lithium as described herein acts on inflammation, acute pain and chronic pain. Parenteral injection into the joint and particularly around the outside of the joint and/or bursa may also provide pain relief. An intradermal or subcutaneous injection of lithium as described herein may arrest an acute gout attack, in particular. Lithium, in any combination or formulation, is able to penetrate to the capsular, tendinous, and periarticular structures in sufficient concentration to produce the desired effect without systemic toxicity.

“Subject” as used herein may be a patient. In some embodiments, the subject is a human; however, a subject of this disclosure can include an animal subject, particularly mammalian subjects such as canines, felines, bovines, caprines, equines, ovines, porcines, rodents (e.g. rats and mice), lagomorphs, primates (including non-human primates), etc., including domesticated animals, companion animals and wild animals for veterinary medicine, treatment or pharmaceutical drug development purposes.

The subjects relevant to this disclosure may be male or female and may be any species and of any race or ethnicity, including, but not limited to, Caucasian, African-American, African, Asian, Hispanic, Indian, etc., and combined backgrounds. The subjects may be of any age, including newborn, neonate, infant, child, adolescent, adult, and geriatric. In some embodiments, the subject has or is at risk for suffering from an inflammatory joint disease. In certain embodiments, the subject has or is at risk for suffering from arthritis, tendonitis, bursitis, inflammation of the ligament, synovitis, gout, or systemic lupus erythematosus. In some embodiments, the subject has or is at high risk for suffering from gout. In certain embodiments, the subject is obese; suffers from high blood pressure, diabetes, metabolic syndrome, heart and/or kidney disease; uses thiazide diuretics, aspirin and/or anti-rejection drugs; or has a family history of gout. In some embodiments, the subject is experiencing pain or at risk for experiencing pain as described herein. In particular embodiments, the subject has experienced or is at risk for opioid addiction.

Embodiments of the present inventive concept further provide kits for the prevention or treatment of inflammatory conditions, inflammatory joint diseases and also pain in a subject as described herein. As used herein, “kit” refers to an assembly of components packaged together with optional instructions (written, audio or visual) regarding how to use the components of the kit. According to embodiments of the present invention, the kits may include all components of the elements described herein for treating inflammatory conditions, inflammatory joint diseases or treating acute and chronic pain, or a subset of the elements in any combination. The kit may include packaging materials that may maintain the components and can be composed of material commonly used for such purposes (e.g., paper, corrugated fiber, glass, plastic, foil, ampules, vials, tubes, bottles, syringes, etc.). In addition to including lithium as an active pharmaceutical ingredient, a kit may also contain an anti-gout agent and/or a pain management agent. In some embodiments, the kits are sterile.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner to make compounds of the disclosure by other methods.

### Example 1

### Gout Formula 1

Gout Formula 1 was compounded as a pump spray in deionized water. The intended goal of treatment was to control the pain and stop (or reverse) the chemical and cellular events and micropathophysiology of the acute gout attack. Each component of the formulation was designed to have a specific role in the acute gouty cascade. This formulation was applied topically by three (3) subjects resulting in some relief in subjective painful symptoms.

1) Lidocaine 5% (optional)

2) Ketoprofen 5% (optional

3) Colchicine 1% (optional)

4) Allopurinol 3% (optional)

5) Lithium Carbonate 2%

6) DMSO Spray Base

7) Steroid (optional)

8) Benadryl (optional)

9) Uricase (optional)

### Example 2

### Gout Formula 2

Lithium carbonate syrup 8 MEQ/5 ml was topically applied to a painful inflamed bunion of one subject.

### Example 3

### Gout Formula 2

In this formulation, 2% lithium carbonate capsules were gradually dissolved in a witch hazel base. The gelatin of the capsules added a slight viscosity to the solution; acting as a very thin lotion. This was more easily applied topically and tolerated by the 18 patients. The relief of pain and swelling was noticeable and appreciated by many patients, and refills were requested and filled PRN. Serum lithium levels drawn in at least three (3) subjects showed no detectable serum concentration (at 0.8 to 1.2 mEq/L range).

### Example 4

### Gout Formula 4

Commercially available white lithium grease in a cream or spray formulation was used by subjects and applied topically to areas of pain suffered by gout patients. Favorable results were achieved with continued relief of pain in several gout patient with one patient presenting bullous pemphigous.

### Example 5

### Gout Formulas 5 and 6

LJP-Formula 5
2% Lithium carbonate (prepared from tablets, crushed)
10% DMSO
Cream Base 1.5 g/pump metered dose

LJP-Rescue Formula 6
2% Lithium carbonate (prepared from tablets, crushed)
2% Lidocaine
10% DMSO
Cream base 1.5 g/pump metered dose

Topical lithium carbonate relieves pain. This case study relates to the presentation of a single case of a 61-year-old female with painful peripheral neuropathy. The patient had a history of low back pain, opioid dependence and IDDM and developed a gradual neuromuscular decline. Lower extremity weakness/atrophy, numbness, and pain increased to a point of instability in gait. She was unable to drive without placing herself and others at risk. Several falls, fractures, and head trauma occurred, without CT evidence of subdural hemorrhage or lesion. The patient next developed Hashimoto's disease, with tonic-clonic seizures, and was EMS transported and hospitalized, never to walk again. Transverse myelitis, compression fracture of the spine, herniated disc, and spinal stenosis, and cauda equinus syndrome were all later diagnosed. She requires 24-hour skilled nursing care and medical management. After one year in skilled nursing, she was allowed to fall during personal care; and sustained pilon fractures of both femurs just above the knee. These were reduced and fixated surgically by orthopaedics. The patient had severe painful neuropathy, allodynia, and hyperalgesia in the knees, ankle, and feet. The patient described the pain as burning, ants crawling, squeezing pressure, and also an occasional shooting pain. She was unable to wear socks; and light touch elicited excruciating pain. Keppra, Elavil, Duloxetine, and Gabapentin are included in her pharmacopeia. She takes no opioids. Patient is currently awaiting testing to R/O Bartonella and Babesia involvement as a possible causation for transverse myelitis and other neuromuscular symptoms.

Application of LJP—Formula 5 daily to the feet immediately brought relief and resolution of pain. After 2-3 days, the patient was able to tolerate application w/o squirming from allodynia. The patient remains pain-free with single daily application. On two occasions, the patient skipped application for a few days; and pain returned. Upon resuming daily application of 2% lithium carbonate and 10% DMSO, the pain once again abated. LJP-Rescue Formula 6 was not needed. However, LJP-Rescue formula 6 was used to arrest an acute gout attack in a 70-year-old woman; receiving relief after 1 day with 3 applications to the great toe.

### Example 6

### Gout Formula 7

The objective of this study was to evaluate the efficacy of a lithium carbonate formulation (Lithium carbonate, 2%/DMSO, 10%/Acetic acid, 3% in water) on gouty inflammation in Sprague Dawley (CD®) male rats. Endpoints included ankle swelling, pain, and synovial fluid cytokines.

Monosodium urate (MSU) injection caused significant swelling and pain at 4 and 8 hours in all animals, compared to untreated or PBS-injected animals, confirming the MSU-induced gouty inflammation.

A slight reduction (12% to 17%) in swelling at 4 and 8 hours was observed for animals treated with Lithium. Though not statistically significant, the reduction in swelling was also accompanied by 33% reduction in the Pain Index at 8 hrs. In the synovial fluid, Lithium treatment decreased KC-GRO and increased in IL-1b and IL-6.

| | |
|---|---|
| Identification: | Lithium Carbonate, 2%/DMSO, 10%/Acetic acid, 3% in |
| | water |
| Dose volume: | 1.0 mL |
| Route: | Dermal application to left ankle/foot area (Grp 3 rats) |

### Animals

Sprague Dawley [Crl:CD(SD)] male rats, purchased from Charles River were acclimated for 3 weeks prior to use. Rats were approximately 9-10 weeks old at the start of the study. Only healthy animals were assigned to the study. The rats were randomized into study groups.

### Food and Water

Animals were provided with Purina 5002 Rodent Chow and access to tap water ad libitum. No contaminants are expected to have been present in dietary materials that are known to be capable of interfering with the study.

### Housing and Identification

Rats were housed (up to 3 per cage) in individually ventilated shoebox-style plastic cages containing BioFresh bedding, with rat huts and nylon bones as enrichment. Each cage was labeled with the study number, animal number, and treatment group.

### Environmental Control

Animals were maintained on approximately 12 hour day and 12 hour night cycles. Temporary adjustments to the light/dark cycles were permitted to accommodate activities within the housing room. Animal room temperature and relative humidity (RH) were monitored and recorded in the study file. Notwithstanding some acceptable minor fluctuations of short duration, the animal room temperature and RH were within laboratory SOP specified ranges of 20-26° C. and 30-70% RH.

### Study Outline

| | Right | Left | Left Ankle ONLY |
|---|---|---|---|
| | Ankle | Ankle | (Topical Treatment |
| Group | (IA) | (IA) | post IA)¹ |
| 1 (Model) | None | MSU, 1.25 mg | None |
| | | in PBS (50 uL) | |
| 2 (Vehicle) | PBS (50 uL) | MSU, 1.25 mg | 10% DMSO/3% Acetic |
| | | in PBS (50 uL) | acid |
| 3 (Lithium | PBS (50 uL) | MSU, 1.25 mg | Lithium Carbonate 2% in |
| Formulation) | | in PBS (50 uL) | 10% DMSO/3% Acetic |
| | | | acid |

¹Treatments (Groups 2 and 3 only) will be applied to the ankle/foot area immediately following injection of MSU in left ankle.

### Preparation of MSU

MSU was sterilized by autoclaving. Sterile MSU was resuspended with sterile PBS to prepare a 25 mg/mL suspension.

### Injection of MSU Crystals for Gouty Inflammation

All rats received an IA injection of 1.25 mg/50 uL MSU in the left ankle. Groups 2 and 3 only received an IA injection of sterile PBS in the right ankle. The right ankle of Group 1 rats did not receive any injection.

Dosing with Test and Control Articles

Following the MSU/PBS IA injections, rats from the appropriate groups (Groups 2 and 3 only) also received a dermal application of 0.1 mL of the appropriate treatment to their left ankle/foot area. No treatment was applied to the right ankle/foot. Residual test and control articles will be discarded, and disposition documented in the study records.

### Measurement of Ankle Swelling

Animals were anesthetized with isoflurane per laboratory SOPs, and ankle measurements (both ankles/all rats) were taken with a caliper prior to injection (0 hr), and at 4 hrs and 8 hrs post-injection.

### Measurement of Pain and Clinical Observations

Clinical observations included increased attention by the animal to the injected foot as an indication of pain, if applicable. For evaluation of Pain Index, the animals were moved individually to an empty cage/plexiglass surface to evaluate willingness to put weight on paw of affected ankle at 0, 4, and 8 hours post-MSU injection. The following scoring system was used:
0=normal paw pressure, equal weight on both hind paws;
1=slightly reduced paw pressure (paw is completely on the floor but toes are not spread);
2=moderately reduced paw pressure (foot curled with only some parts of the foot lightly touching the floor);
3=severely reduced paw pressure (foot elevated completely).

### Terminal Procedures

Animals were euthanized with CO₂ per laboratory SOPs and current AVMA guidelines. Sterile PBS (0.5 mL) was injected in the synovial cavity of the ankle and synovial fluid collected and frozen at <−70 C for cytokine analysis. Synovial fluid was collected from both ankles for all rats. Following synovial fluid collection, the fur/skin was removed and the ankle preserved for possible future histopathology evaluation.

### Cytokine Evaluations

Synovial fluid collected from the ankles of all rats was evaluated for cytokines (IL-1β, IL-6, KC-GRO, and TNFα) by multiplex ELISA (Mesoscale Discovery). Samples were removed from the freezer, thawed, and diluted for analysis. The ELISA/multiplex methods were performed as per manufacturer's instructions and laboratory qualification.

All samples were evaluated in duplicate wells and mean values were reported. Any samples that were BLQ (below the limit of quantitation) of the kits were assigned a value equivalent to ½ of the LLOQ (lower limit of quantitation) for calculation of group means.

### Data and Statistical Analysis

Calculation of group means and standard errors (SE) were performed using GraphPad Prism v.6. Differences in treatment to MSU-injected ankles were evaluated by one-way ANOVA.

### Results

### Ankle Swelling and Pain Index

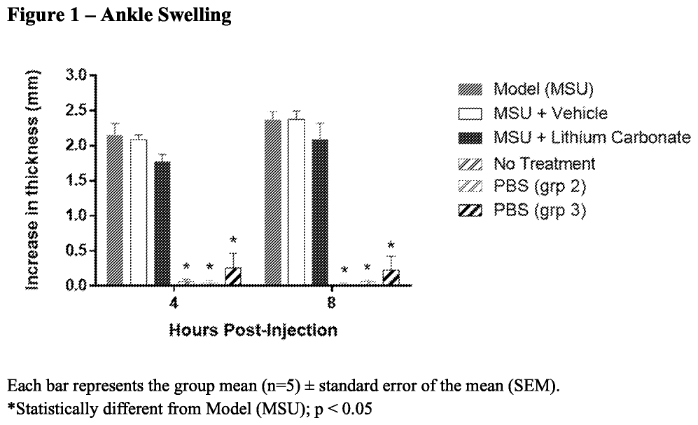

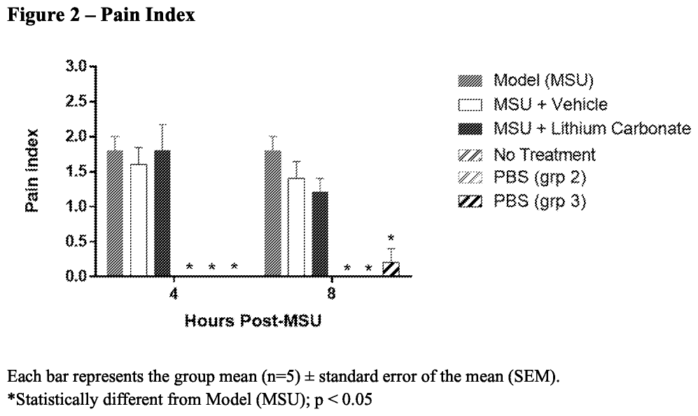

Group summary data for ankle swelling are presented in Table 1 and FIG. 1, and Pain Index are presented in Table 2 and FIG. 2. Individual animal data are presented in Table 7 for ankle pain and in Table 8 for pain index.

The MSU injection in the left ankle of animals in Group 1 caused significant swelling and pain at 4 and 8 hours in all animals, compared to untreated (Group 1, right ankle) or PBS-injected (Groups 2 and 3, right ankle), confirming the MSU-induced gouty inflammation.

A slight reduction (12% to 17%) in swelling at 4 and 8 hours was observed for animals treated with Lithium. Though not statistically significant, the reduction in swelling was also accompanied by 33% reduction in the Pain Index at 8 hrs.

### Synovial Fluid Cytokines

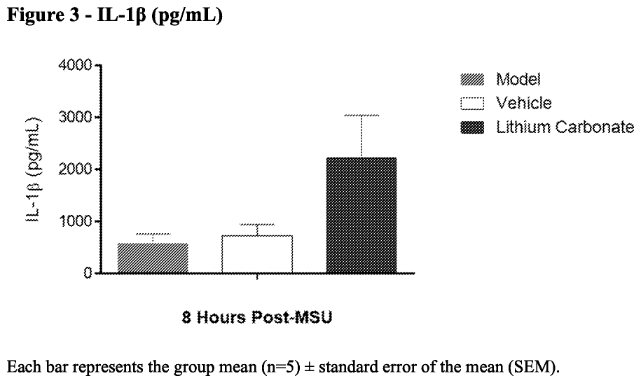

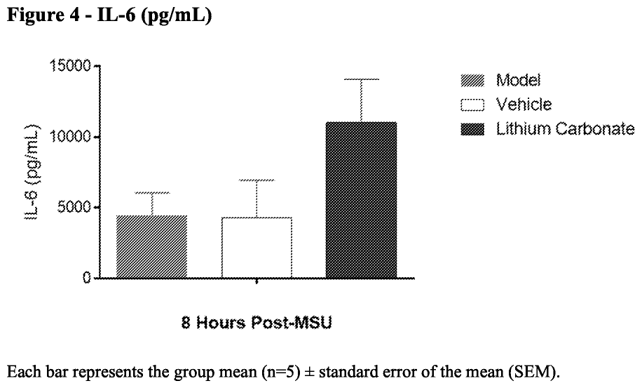

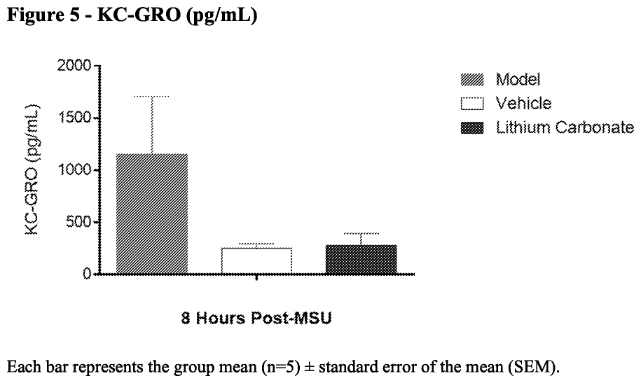

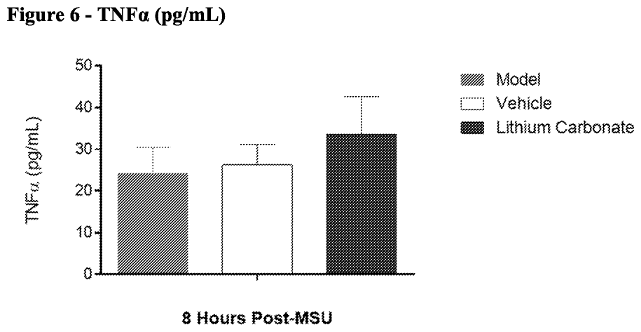

Individual animal data are presented in Table 9 Group summary tables and data for cytokines in the Model and treated MSU-injected ankles are presented for IL-1β (Table 3, FIG. 3), IL-6 (Table 4, FIG. 4), KC-GRO (Table 5, FIG. 5), and TNF-α (Table 6, FIG. 6).

There was no effect of Vehicle or Lithium carbonate treatment on TNF-α in the synovial fluid of animals treated with Vehicle or Lithium. Though not statistically significant, both treatments caused approximately 4-fold reduction in KC-GRO, compared to the Group 1 Model/MSU. Lithium carbonate treatment also increased IL-1β and IL-6 (3.8× and 2.5× respectively), compared to Group 1 Model/MSU animals.

Though not statistically significant, treatment of rats with dermal application of lithium carbonate resulted in small decreases in ankle swelling (4 and 8 hours) and pain (8 hours). In the synovial fluid, lithium treatment decreased KC-GRO and increased in IL-1β and IL-6.

### TABLES

TABLE 1
Ankle Swelling

| | | 4 hrs Post-Injection | | | 8 hrs Post-Injection | | |
|---|---|---|---|---|---|---|---|
| Group | Injection + Treatment | Mean | SEM | N | Mean | SEM | N |
| 1 (left ankle) | Model (MSU) only | 2.14 | 0.18 | 5 | 2.36 | 0.12 | 5 |
| 2 (left ankle) | MSU + Vehicle | 2.08 | 0.07 | 5 | 2.37 | 0.12 | 5 |
| 3 (left ankle) | MSU + Lithium | 1.77 | 0.11 | 5 | 2.08 | 0.24 | 5 |
| 1 (right ankle) | No treatment | 0.06 | 0.04 | 5 | 0.02 | 0.02 | 5 |
| 2 (right ankle) | PBS injection only | 0.04 | 0.04 | 5 | 0.06 | 0.02 | 5 |
| 3 (right ankle) | PBS injection only | 0.25 | 0.21 | 5 | 0.23 | 0.19 | 5 |

TABLE 2
Pain Index

| | | 4 hrs Post-Injection | | | 8 hrs Post-Injection | | |
|---|---|---|---|---|---|---|---|
| Group | Injection + Treatment | Mean | SEM | N | Mean | SEM | N |
| 1 (left ankle) | Model (MSU) only | 1.80 | 0.200 | 5 | 1.80 | 0.200 | 5 |
| 2 (left ankle) | MSU + Vehicle | 1.60 | 0.245 | 5 | 1.40 | 0.245 | 5 |
| 3 (left ankle) | MSU + Lithium | 1.80 | 0.374 | 5 | 1.20 | 0.200 | 5 |
| 1 (right ankle) | No treatment | 0.00 | 0.000 | 5 | 0.00 | 0.000 | 5 |
| 2 (right ankle) | PBS injection only | 0.00 | 0.000 | 5 | 0.00 | 0.000 | 5 |
| 3 (right ankle) | PBS injection only | 0.00 | 0.000 | 5 | 0.200 | 0.200 | 5 |

TABLE 3
IL-1β (pg/mL)

8 hrs Post-Injection

| Group | Injection + Treatment | Mean | SEM | N |
|---|---|---|---|---|
| 1 (left ankle) | Model (MSU) only | 579 | 187 | 5 |
| 2 (left ankle) | MSU + Vehicle | 730 | 213 | 5 |
| 3 (left ankle) | MSU + Lithium | 2229 | 814 | 5 |

TABLE 4
IL-6 (pg/mL)

8 hrs Post-Injection

| Group | Injection + Treatment | Mean | SEM | N |
|---|---|---|---|---|
| 1 (left ankle) | Model (MSU) only | 4474 | 1568 | 5 |
| 2 (left ankle) | MSU + Vehicle | 4295 | 2640 | 5 |
| 3 (left ankle) | MSU + Lithium | 11022 | 3065 | 5 |

TABLE 5
KC-GRO (pg/mL)

8 hrs Post-Injection

| Group | Injection + Treatment | Mean | SEM | N |
|---|---|---|---|---|
| 1 (left ankle) | Model (MSU) only | 1154 | 554 | 5 |
| 2 (left ankle) | MSU + Vehicle | 249 | 46.1 | 5 |
| 3 (left ankle) | MSU + Lithium | 280 | 115 | 5 |

TABLE 6
TNF-α (pg/mL)

8 hrs Post-Injection

| Group | Injection + Treatment | Mean | SEM | N |
|---|---|---|---|---|
| 1 (left ankle) | Model (MSU) only | 24.1 | 6.28 | 5 |
| 2 (left ankle) | MSU + Vehicle | 26.2 | 4.95 | 5 |
| 3 (left ankle) | MSU + Lithium | 33.6 | 9.00 | 5 |

TABLE 7
Individual Animal Data, Ankle Swelling

| | | Pre-injection Ankle | | 4 Hr Past Ankle | | 8 Hr Post Ankle | | 4 Hr Increase from | | 8 Hr Increase from | |
|---|---|---|---|---|---|---|---|---|---|---|---|
| | | Measurement | | Measurement | | Measurement | | pre-injection | | pre-injection | |
| Group | Animal | (mm) | | (mm) | | (mm) | | (mm) | | (mm) | |
| # | # | Left (L) | Right (R) | MSU (L) | PBS (R)* | MSU (L) | PBS (R)* | MSU (L) | PBS(R) | MSU (L) | PBS (R) |
| 1 | 6 | 10.40 | 10.00 | 12.10 | 10.18 | 12.60 | 10.00 | 1.70 | 0.18 | 2.20 | 0.00 |
| Model | 7 | 10.00 | 10.00 | 12.10 | 10.00 | 12.70 | 10.10 | 2.10 | 0.00 | 2.70 | 0.10 |
| | 8 | 10.10 | 10.00 | 12.89 | 10.00 | 12.30 | 10.00 | 2.79 | 0.00 | 2.20 | 0.00 |
| | 9 | 10.00 | 10.00 | 12.10 | 10.00 | 12.60 | 10.00 | 2.10 | 0.00 | 2.60 | 0.00 |
| | 10 | 10.00 | 10.00 | 12.00 | 10.10 | 12.10 | 10.00 | 2.00 | 0.10 | 2.10 | 0.00 |
| 2 | 11 | 10.00 | 10.00 | 12.10 | 10.00 | 12.00 | 10.00 | 2.10 | 0.00 | 2.00 | 0.00 |
| Veh. | 12 | 10.00 | 9.90 | 12.20 | 10.10 | 12.45 | 10.00 | 2.20 | 0.20 | 2.45 | 0.10 |
| | 13 | 10.00 | 10.00 | 11.80 | 10.00 | 12.70 | 10.10 | 1.80 | 0.00 | 2.70 | 0.10 |
| | 14 | 10.00 | 10.00 | 12.10 | 10.00 | 12.20 | 10.10 | 2.10 | 0.00 | 2.20 | 0.10 |
| | 15 | 10.00 | 10.00 | 12.20 | 10.00 | 12.50 | 10.00 | 2.20 | 0.00 | 2.50 | 0.00 |
| 3 | 16 | 10.00 | 10.00 | 11.50 | 11.10 | 12.60 | 11.00 | 1.50 | 1.10 | 2.60 | 1.00 |
| Lithium | 17 | 10.00 | 10.00 | 12.10 | 10.00 | 12.70 | 10.00 | 2.10 | 0.00 | 2.70 | 0.00 |
| | 18 | 10.00 | 9.85 | 11.60 | 10.00 | 11.60 | 10.00 | 1.60 | 0.15 | 1.60 | 0.15 |
| | 19 | 10.00 | 10.00 | 11.75 | 10.00 | 11.60 | 10.00 | 1.75 | 0.00 | 1.60 | 0.00 |
| | 20 | 10.00 | 10.00 | 11.90 | 10.00 | 11.90 | 10.00 | 1.90 | 0.00 | 1.90 | 0.00 |

*PBS in the right ankle only for Groups 2 and 3. Group 1 had no injection to right ankle.

TABLE 8
Individual Animal Data, Pain Index

| | | Pre - Paw Pressure | | 4 hour - Paw | | 8 hour - Paw | |
|---|---|---|---|---|---|---|---|
| Group | Animal | Scoring | | Pressure Scoring | | Pressure Scoring | |
| # | # | Left (L) | Right (R) | MSU (L) | PBS (R)* | MSU (L) | PBS (R)* |
| 1 | 6 | 0 | 0 | 2 | 0 | 2 | 0 |
| Model | 7 | 0 | 0 | 2 | 0 | 2 | 0 |
| | 8 | 0 | 0 | 1 | 0 | 1 | 0 |
| | 9 | 0 | 0 | 2 | 0 | 2 | 0 |
| | 10 | 0 | 0 | 2 | 0 | 2 | 0 |
| 2 | 11 | 0 | 0 | 2 | 0 | 2 | 0 |
| Veh. | 12 | 0 | 0 | 2 | 0 | 1 | 0 |
| | 13 | 0 | 0 | 1 | 0 | 1 | 0 |
| | 14 | 0 | 0 | 1 | 0 | 2 | 0 |
| | 15 | 0 | 0 | 2 | 0 | 1 | 0 |
| 3 | 16 | 0 | 0 | 1 | 0 | 1 | 1 |
| Lithium | 17 | 0 | 0 | 3 | 0 | 2 | 0 |
| | 18 | 0 | 0 | 2 | 0 | 1 | 0 |
| | 19 | 0 | 0 | 1 | 0 | 1 | 0 |
| | 20 | 0 | 0 | 2 | 0 | 1 | 0 |

*PBS in the right ankle only for Groups 2 and 3. Group 1 had no injection to right ankle.

TABLE 9
Individual Animal Data, Cytokines

| | | | | IL-1β | IL-6 | KC/GRO | TNF-α |
|---|---|---|---|---|---|---|---|
| Animal | Ankle Joint | Group | Treatment | (pg/mL) | (pg/mL) | (pg/mL) | (pg/mL) |
| 6 | Right Ankle | 1 | Untreated | BLQ | BLQ | BLQ | BLQ |
| 7 | Right Ankle | 1 | Untreated | BLQ | BLQ | 32.4 | BLQ |
| 8 | Right Ankle | 1 | Untreated | BLQ | BLQ | BLQ | BLQ |
| 9 | Right Ankle | 1 | Untreated | BLQ | BLQ | BLQ | BLQ |
| 10 | Right Ankle | 1 | Untreated | BLQ | BLQ | BLQ | BLQ |
| 11 | Right Ankle | 2 | PBS | 527 | 644 | 266 | 25.8 |
| 12 | Right Ankle | 2 | PBS | 838 | 2690 | 443 | 29.7 |
| 13 | Right Ankle | 2 | PBS | 466 | 674 | 101 | 9.61 |
| 14 | Right Ankle | 2 | PBS | 893 | 5242 | 128 | 19.2 |
| 15 | Right Ankle | 2 | PBS | 739 | 1502 | 72.8 | 23.3 |
| 16 | Right Ankle | 3 | PBS | 223 | 3370 | 61.1 | BLQ |
| 17 | Right Ankle | 3 | PBS | 744 | 1330 | 111 | 9.99 |
| 18 | Right Ankle | 3 | PBS | 265 | 314 | 36.6 | BLQ |
| 19 | Right Ankle | 3 | PBS | 431 | 2296 | 54.9 | BLQ |
| 20 | Right Ankle | 3 | PBS | BLQ | 676 | 27.9 | BLQ |
| 6 | Left Ankle | 1 | MSU | 594 | 7800 | 816 | 31.1 |
| 7 | Left Ankle | 1 | MSU | 1178 | 8537 | 3280 | 38.4 |
| 8 | Left Ankle | 1 | MSU | 364 | 2631 | 458 | 14.7 |
| 9 | Left Ankle | 1 | MSU | BLQ | 518 | 152 | BLQ |
| 10 | Left Ankle | 1 | MSU | 706 | 2884 | 1066 | 32.2 |
| 11 | Left Ankle | 2 | MSU + Veh. | 431 | 505 | 291 | 30.1 |
| 12 | Left Ankle | 2 | MSU + Veh. | 466 | 1840 | 287 | 18.1 |
| 13 | Left Ankle | 2 | MSU + Veh. | 1563 | 2729 | 377 | 40.6 |
| 14 | Left Ankle | 2 | MSU + Veh. | 699 | 14759 | 170 | 12.5 |
| 15 | Left Ankle | 2 | MSU + Veh. | 493 | 1643 | 120 | 29.9 |
| 16 | Left Ankle | 3 | MSU + Lithium | 800 | 5890 | 117 | 14.3 |
| 17 | Left Ankle | 3 | MSU + Lithium | 3586 | 17496 | 716 | 45.2 |
| 18 | Left Ankle | 3 | MSU + Lithium | 1501 | 2610 | 166 | 44.0 |
| 19 | Left Ankle | 3 | MSU + Lithium | 4691 | 11099 | 306 | 54.7 |
| 20 | Left Ankle | 3 | MSU + Lithium | 567 | 18013 | 92.7 | 10.0 |

| | | | | | |
|---|---|---|---|---|---|
| BLQ | < | 102 | 96.9 | 21.7 | 9.10 |
| ½ BLQ | (pg/mL) used for calculation of group mean | 51.0 | 48.5 | 10.9 | 4.55 |

### Example 7

### Validation Study with Human Cell-Based Screening

The purpose of this study is to validate the use of lithium, for example, lithium carbonate, to treat neuroinflammation and pain in selected targets and provide for the development of a human cellular translational platform for the study of both the diffusion and efficacy of any topically-applied or injected API.

The goal of this study will be to simulate tissue layers in the anatomical area of interest; simulate/study the desired micropathophysiology of the normal/abnormal tissue model and create a human-biosimilar model with 3-D printing of the epidermis and underlying tissues which could be manipulated as needed to match the anatomy of the area of interest. A Neuropathic Pain Model (epidermis, dermal fibroblasts and nociceptors); Muscle Pain Model (epidermis, dermal fibroblasts, nociceptors, adipocytes, myocytes and myelinated neurons); Joint Pain Model (epidermis, dermal fibroblasts, nociceptors, synovial fibrocytes, chondrocytes and osteocytes); and a Spinal Pain Model (epidermis, dermal fibroblasts, nociceptors, myocytes, DRG cells and glial cells) will be established.

This model has great potential with a 3-D bio printed tri-layered matrix cell of keratinocytes, myocytes, and DRG cells/glial cells. Chronic neuromuscular back pain may be treated by a pain management specialist, anesthesia, or surgeon after epidural corticosteroid or intervertebral lithium injection.

Human cell-based screening (HCBS) may minimize the hurdles that may be seen with a failed proof of concept or clinical trial. The impact of any delay equates to lives, functionality, and productivity lost to chronic pain, and opioid addiction. The ability to print and simulate the BBB is critical in any pharmaceutical research and development endeavor; however, the ability to recreate the epidermal barrier and the underlying strata of the human anatomy may prove just as important. The use of HCBS to validate the use of lithium carbonate as a topical formulation may provide rapid entry of an affordable adjunct to managing chronic pain and neuroinflammation of many etiologies, while simultaneously developing novel physiologic and anatomically specific targeted 3D HCBS models of nociception.

The present inventive concept may increase solubility of and allow dissolution and elimination of crystals. The present inventive concept may act to decrease the inflammatory response in the involved tissues. The present inventive concept may decrease motility and recruitment of monocytes and macrophages at the CXCL1/CXCL2 level. The present inventive concept may stabilize and have protective functions on the phospholipids of the cartilage, joint, capsule, and tendinous structures. The present inventive concept may have implications in the treatment of chronic back pain.

The present inventive concept may substitute for magnesium in many pathways. The hydrated ion employed in the present invention is smaller; and may be particularly preferred in small enzyme pockets. The present inventive concept may act via Na/Ca/K ion channels as a smaller, more reactive molecule. The present inventive concept may interfere with K+ efflux, delaying repolarization of nerve. The present inventive concept may extend the duration and lower the effective dose of local anesthetics in subcutaneous and peripheral nerves/nociceptors. The present inventive concept may affect glutamate and substance P signaling in peripheral nerves. The present inventive concept may decrease the hyperactivity of peripheral ectopic nociceptor firing. The present inventive concept may act in the periphery to decrease the upregulation of neurotransmitters within the dorsal root ganglion. The present inventive concept may affect the afferent axon at multiple levels. The present inventive concept may act via direct and indirect inhibition of GSK-3 in peripheral tissues. The present inventive concept may have implications for affecting neuroinflammation. The present inventive concept may provide neuroprotective benefits and may allow for the remyelination of degenerated peripheral nerves commonly seen in small fiber neuropathy. This may have implications for treating the localized pain, inflammation, crystal deposition, tophus, and joint and tissue destruction associated with the crystalline arthropathies. This may also have implications for treating neuroinflammation. This may have further implications for mitigation of the painful symptoms and pathology associated with, but not limited to, the following: diabetic peripheral neuropathy, allodynia, opioid induced hyperalgesia, fibromyalgia, complex regional pain syndrome (CRPS or RSD) peripheral nerve injury, post-herpetic neuralgia, trigeminal neuralgia, Morton's Neuroma, painful nerve entrapment, & postoperative pain. The present inventive concept may produce desired effects on peripheral targets, without the undesirable CNS and systemic side effects. Moreover, the present inventive concept may reduce the morbidity and/or disability associated with inflammatory arthropathies and/or pain syndromes. The present inventive concept may have applications in the treatment of acute spinal injury/trauma.

Accordingly, the present inventive concept may have implications for the treatment of acute and chronic pain; thereby addressing the challenges that the opioid epidemic has brought to our society and may capture a niche in the market which is currently not met.

Although the present inventive concept has been explained in relation to various embodiments, it is to be understood that many other possible modifications and variations can be made without departing from the spirit and scope of the invention. The following claim is provided to ensure that the present application meets all statutory requirements as a priority application in all jurisdictions and shall be construed as setting forth the scope of the present inventive concept.

1. A method of treating an inflammatory condition or inflammatory joint disease in a subject, the method comprising administering to the subject an effective amount of a composition comprising a lithium agent in a pharmaceutically acceptable carrier.

2. The method of claim 1, wherein the inflammatory joint disease is arthritis, tendonitis, bursitis, inflammation of the ligament, synovitis, or gout.

3. The method of claim 1, wherein the method further comprises topically applying the composition comprising the lithium agent in a pharmaceutically acceptable carrier to an area selected from the group consisting of a knee, elbow, wrist, finger, toe, foot, ankle, hip, shoulder, spine or combination thereof.

4. The method of claim 1, wherein the method further comprises injecting the composition comprising the lithium agent in a pharmaceutically acceptable carrier into or around an area selected from the group consisting of a knee, elbow, wrist, finger, toe, foot, ankle, hip, shoulder, spine or combination thereof.

5. The method of claim 2, wherein the gout is one of more of acute gout, chronic gout, moderate gout, severe gout, recurrent gout, refractory gout, quiescent gout, gouty arthritis, hyperuricaemia, gout-related cardiovascular disorders, Lesch-Nyhan syndrome, Kelley-Seegmiller syndrome, kidney disease, kidney stones, kidney failure, joint inflammation, inflammatory joint disease, arthritis, osteoarthritis, rheumatoid arthritis and psoriatic arthritis, crystalline arthropathy, bursitis, tendinopathy urolithiasis, plumbism, hyperparathyroidism, psoriasis or sarcoidosis.

6. The method of claim 2, wherein the number, duration, frequency or intensity of a gout flare experienced by a subject is decreased during initiation or maintenance administration of the composition comprising lithium in a pharmaceutically acceptable carrier.

7. The method of claim 1, wherein the lithium agent is elemental lithium, a lithium mimetic, lithium acetate, lithium aluminate, lithium aluminum hydride, lithium amide, lithium aspartate, lithium azide, lithium beryllide, lithium bis(trifluoromethanesulfonyl)imide, lithium bis(trimethylsilyl)amide, lithium borate, lithium borohydride, lithium bromide, lithium carbide, lithium carbonate, lithium chlorate, lithium chloride, lithium citrate, lithium cobalt oxide, lithium cyanide, lithium diisopropylamide, lithium disilicate, lithium fluoride, lithium hexafluorogermanate, lithium hexafluorophosphate, lithium hydride, lithium hydroxide, lithium hypochlorite, lithium imide, lithium iodate, lithium iodide, lithium iridate, lithium iron phosphate, lithium lactate, lithium metaborate, lithium metasilicate, lithium methoxide, lithium molybdate, lithium molybdenum purple bronze, lithium monoxide anion, lithium nickel cobalt aluminum oxides, lithium nickel manganese cobalt oxides, lithium niobate, lithium nitrate, lithium nitride, lithium nitrite, lithium orotate, lithium orthosilicate, lithium oxide, lithium perchlorate, lithium peroxide, lithium platinate, lithium polonide, lithium ruthenate, lithium salicylate, lithium selenide, lithium stearate, lithium succinate, lithium sulfate, lithium sulfide, lithium sulfite, lithium superoxide, lithium tantalate, lithium tetrachloroaluminate, lithium tetrafluoroborate, lithium tetrahydridogallate, lithium tetrakis(pentafluorophenyl)borate, lithium tetramethylpiperidide, lithium titanate, lithium triborate, lithium triethylborohydride, lithium triflate or lithium tungstate.

8. The method of claim 7, wherein the lithium agent is lithium carbonate, lithium citrate, lithium salicylate or lithium lactate.

9. The method of claim 1, wherein the pharmaceutically acceptable carrier is dimethyl sulfoxide (DMSO).

10. The method of claim 1, wherein the method further comprises concomitant administration to the subject of an effective amount of an anti-gout agent or a pain management agent.

11. The method of claim 10, wherein the concomitant administration comprises concurrent administration.

12. The method of claim 10, wherein the concomitant administration comprises sequential administration.

13. The method of claim 10, wherein the anti-gout agent or the pain management agent is a nonsteroidal anti-inflammatory drug (NSAID), an opiate, a local anesthetic, a steroid, a xanthine oxidase inhibitor, a uricosuric drug, or an alkaloid drug.

14. The method of claim 13, wherein the anti-gout agent or a pain management agent is allopurinol, probenecid, benzbromarone, sulfinpyrazone, febuxostat, lesinurad, pegloticase, indomethecin or colchicine.

15. A method of treating acute or chronic pain, the method comprising administering topically or parenterally an effective amount a composition comprising a lithium agent in a pharmaceutically acceptable carrier to a subject in need thereof.

16. The method of claim 15, wherein the pain is selected from the group consisting of pain caused by inflammation of joints, tendons, nerves, muscle, and other soft tissues; arthritic pain; back pain; headache pain; nociceptive pain; pain caused by cancer; and neuropathic pain.

17. The method of claim 15, wherein the pain is complex regional pain syndrome (CRPS), allodynia, fibromyalgia, diabetic neuropathy, chemotherapy pain, shingles, trigeminal neuralgia or thoracic post herpetic neuralgia.

18. The method of claim 15, wherein the lithium agent is elemental lithium, a lithium mimetic, lithium acetate, lithium aluminate, lithium aluminum hydride, lithium amide, lithium aspartate, lithium azide, lithium beryllide, lithium bis(trifluoromethanesulfonyl)imide, lithium bis(trimethylsilyl)amide, lithium borate, lithium borohydride, lithium bromide, lithium carbide, lithium carbonate, lithium chlorate, lithium chloride, lithium citrate, lithium cobalt oxide, lithium cyanide, lithium diisopropylamide, lithium disilicate, lithium fluoride, lithium hexafluorogermanate, lithium hexafluorophosphate, lithium hydride, lithium hydroxide, lithium hypochlorite, lithium imide, lithium iodate, lithium iodide, lithium iridate, lithium iron phosphate, lithium lactate, lithium metaborate, lithium metasilicate, lithium methoxide, lithium molybdate, lithium molybdenum purple bronze, lithium monoxide anion, lithium nickel cobalt aluminum oxides, lithium nickel manganese cobalt oxides, lithium niobate, lithium nitrate, lithium nitride, lithium nitrite, lithium orotate, lithium orthosilicate, lithium oxide, lithium perchlorate, lithium peroxide, lithium platinate, lithium polonide, lithium ruthenate, lithium salicylate, lithium selenide, lithium stearate, lithium succinate, lithium sulfate, lithium sulfide, lithium sulfite, lithium superoxide, lithium tantalate, lithium tetrachloroaluminate, lithium tetrafluoroborate, lithium tetrahydridogallate, lithium tetrakis(pentafluorophenyl)borate, lithium tetramethylpiperidide, lithium titanate, lithium triborate, lithium triethylborohydride, lithium triflate or lithium tungstate.

19. The method of claim 15, wherein the method further comprises concomitant administration to the subject of an effective amount of a pain management agent.

20. A topical or parenteral lithium formulation comprising:
lithium carbonate;
a pharmaceutically acceptable carrier; and
optionally one of a local anesthetic, a nonsteroidal anti-inflammatory drug (NSAID), an alkaloid drug and a xanthine oxidase inhibitor.
